(19) United States
(12) Patent Application Publication
(10) Pub. No.: US 2026/0047824 A1
(43) Pub. Date: Feb. 19, 2026

(54) PANORAMIC ULTRASOUND IMAGE FORMING APPARATUS AND PANORAMIC ULTRASOUND IMAGE FORMING PROGRAM

(71) Applicant: FUJIFILM Corporation, Tokyo (JP)

(72) Inventor: Nobuhiko FUJII, Tokyo (JP)

(73) Assignee: FUJIFILM Corporation, Tokyo (JP)

(21) Appl. No.: 19/296,896

(22) Filed: Aug. 11, 2025

(30) Foreign Application Priority Data
Aug. 13, 2024 (JP) 2024-135138

(51) Int. Cl. A61B 8/00

(52) U.S. Cl. CPC A61B 8/5253; A61B 8/4263; A61B 8/463; A61B 8/5292

(57) ABSTRACT

A probe information acquisition unit acquires a plurality of pieces of positional information indicating a position of an ultrasound probe in a case where a reception signal corresponding to each frame for forming a panoramic ultrasound image is acquired. A combination processing unit disposes a second frame by shifting the second frame with respect to a first frame in a scanning direction based on a difference between a first position, which is a position of the ultrasound probe in a case where the reception signal corresponding to the first frame constituting the panoramic ultrasound image is acquired, and a second position, which is a position of the ultrasound probe in a case where the reception signal corresponding to the second frame constituting the panoramic ultrasound image is acquired, and then combines the first frame and the second frame.

### CROSS REFERENCE TO RELATED APPLICATION

This application claims priority to Japanese Patent Application No. 2024-135138 filed on Aug. 13, 2024 which is incorporated herein by reference in its entirety including the specification, claims, drawings, and abstract.

### BACKGROUND OF THE INVENTION

### 1. Field of the Invention

The present specification discloses improvements in a panoramic ultrasound image forming apparatus and a panoramic ultrasound image forming program.

### 2. Description of the Related Art

An ultrasound diagnostic apparatus that transmits ultrasonic waves from an ultrasound probe toward a subject, receives a reflected wave from the subject in the ultrasound probe, and forms an ultrasound tomographic image representing a cross section of the subject on the basis of a reception signal formed from the reflected wave is known. Since an ultrasound beam is scanned on a scanning surface from the ultrasound probe, the ultrasound tomographic image is an image representing a cross section of the subject on the scanning surface.

In the related art, a panoramic ultrasound image is formed by arranging and combining a plurality of ultrasound tomographic images formed while moving an ultrasound probe in a scanning surface direction (specifically, a scanning direction of an ultrasound beam) in the scanning direction. In the present specification, each ultrasound tomographic image constituting the panoramic ultrasound image is referred to as a “frame”. The panoramic ultrasound image is an ultrasound tomographic image that extends in the scanning direction of the ultrasound beam as compared with a single frame.

For example, JP4299015B discloses an ultrasound image processing apparatus that forms a panoramic ultrasound image by combining a frame n−1 and a frame n which is a frame at a time point subsequent to the frame n−1. Specifically, in the ultrasound image processing apparatus disclosed in JP4299015B, a rectangular reference region is set in the frame n−1, a rectangular region matching the reference region of the frame n−1 is specified from the frame n, the frame n−1 is rotationally moved such that the reference region and the rectangular region match, and then the frame n and the frame n−1 are combined to form a panoramic ultrasound image.

### SUMMARY OF THE INVENTION

As a method of forming a panoramic ultrasound image, in a method of performing registration of two frames (movement or rotation of at least one frame) based on a similarity between frames (for example, a similarity between a reference region of frame n−1 and a rectangular region of frame n, as disclosed in JP4299015B) and then performing the combination, the accuracy of the registration of the frames may be low, and as a result, the image quality of the panoramic ultrasound image may be low.

In addition, in the method of performing the registration of two frames on the basis of the similarity between the frames and then performing the combination, there is a problem in that the amount of calculation for registration of the two frames is increased, that is, the amount of calculation for forming the panoramic ultrasound image is increased.

A purpose of the panoramic ultrasound image forming apparatus disclosed in the present specification is to improve the image quality of the panoramic ultrasound image. Alternatively, the purpose of the panoramic ultrasound image forming apparatus disclosed in the present specification is to reduce the amount of calculation for forming the panoramic ultrasound image.

A panoramic ultrasound image forming apparatus disclosed in the present specification is a panoramic ultrasound image forming apparatus that forms a panoramic ultrasound image by combining a plurality of frames including a first frame obtained by scanning with an ultrasound beam from an ultrasound probe located at a first position toward a subject and a second frame obtained by scanning with the ultrasound beam from the ultrasound probe located at a second position different from the first position toward the subject, comprising: a probe information acquisition unit that acquires positional information indicating a position of the ultrasound probe in a case where a reception signal corresponding to each frame for forming the panoramic ultrasound image is acquired; and a combination processing unit that forms the panoramic ultrasound image by combining the plurality of frames, the combination processing unit being configured to combine the first frame and the second frame by shifting the first frame and the second frame based on a difference between the first position and the second position located in a scanning direction of the ultrasound beam from the first position.

The probe information acquisition unit may further acquire posture information indicating a posture of the ultrasound probe in a case where the reception signal corresponding to each frame for forming the panoramic ultrasound image is acquired, and the combination processing unit may rotate the second frame with respect to the first frame in accordance with a rotation amount corresponding to a difference between a first posture, which is a posture of the ultrasound probe in a case where the ultrasound probe is located at the first position, and a second posture, which is a posture of the ultrasound probe in a case where the ultrasound probe is located at the second position, and may combine the first frame and the second frame.

The combination processing unit may set, based on the first position and the second position, a reference region in a superimposed portion that is a portion of the first frame corresponding to a portion where a scanning surface corresponding to the first frame and a scanning surface corresponding to the second frame overlap each other, and may specify a corresponding region that is a region in the second frame corresponding to the reference region of the first frame and may combine the first frame and the second frame by shifting the first frame and the second frame based on a difference between a position of the reference region and a position of the corresponding region.

The panoramic ultrasound image forming apparatus may further include an intermediate frame generation unit that generates an intermediate frame for interpolating between the first frame and the second frame in the panoramic ultrasound image based on the first frame and the second frame in a case where the second position is separated from the first position by a distance equal to or greater than a first threshold distance, in which the combination processing unit may form the panoramic ultrasound image by combining the first frame, the second frame, and the intermediate frame disposed between the first frame and the second frame.

The panoramic ultrasound image forming apparatus may further include a notification processing unit that notifies an operator in a case where an angle formed between a scanning direction of the ultrasound beam from the ultrasound probe in a case where the ultrasound probe is located at the first position and a direction from the first position toward the second position is equal to or greater than a first threshold angle.

The panoramic ultrasound image forming apparatus may further include a notification processing unit that notifies an operator in a case where an angle formed between a scanning direction of the ultrasound beam in the first posture and a scanning direction of the ultrasound beam in the second posture is equal to or greater than a second threshold angle.

The panoramic ultrasound image forming apparatus may further include a notification processing unit that notifies an operator in a case where the second position is separated from the first position by a distance equal to or greater than a second threshold distance.

The panoramic ultrasound image forming apparatus may further include a camera that images the subject and the ultrasound probe, and a display controller that displays, on a display unit, the panoramic ultrasound image together with a plurality of captured images obtained by imaging the ultrasound probe and the subject, the captured images being arranged in an imaging order, in a case where reception signals corresponding to each of the plurality of frames are acquired.

In addition, a panoramic ultrasound image forming program disclosed in the present specification causes a computer that forms a panoramic ultrasound image by combining a plurality of frames including a first frame obtained by scanning with an ultrasound beam from an ultrasound probe located at a first position toward a subject and a second frame obtained by scanning with the ultrasound beam from the ultrasound probe located at a second position different from the first position toward the subject, to function as: a probe information acquisition unit that acquires positional information indicating a position of the ultrasound probe in a case where a reception signal corresponding to each frame for forming the panoramic ultrasound image is acquired; and a combination processing unit that forms the panoramic ultrasound image by combining the plurality of frames, the combination processing unit being configured to combine the first frame and the second frame by shifting the first frame and the second frame based on a difference between the first position and the second position located in a scanning direction of the ultrasound beam from the first position.

According to the panoramic ultrasound image forming apparatus disclosed in the present specification, it is possible to improve the image quality of the panoramic ultrasound image. Alternatively, with the panoramic ultrasound image forming apparatus disclosed in the present specification, it is possible to reduce the amount of calculation for forming the panoramic ultrasound image.

### BRIEF DESCRIPTION OF THE DRAWINGS

FIG. 1 is a schematic diagram of a configuration of a panoramic ultrasound image forming system according to the present embodiment.

FIG. 2 is a diagram showing an example of a captured image of a camera.

FIG. 3 is a diagram showing a plurality of scanning surfaces corresponding to a plurality of frames.

FIG. 4 is a schematic diagram of a configuration of an ultrasound diagnostic apparatus according to the present embodiment.

FIG. 5 is a diagram showing an angle as posture information.

FIG. 6 is a first diagram showing ultrasound probes located at a first position and a second position.

FIG. 7 is a first diagram showing a first frame and a second frame to be combined.

FIG. 8 is a second diagram showing the ultrasound probes located at the first position and the second position.

FIG. 9 is a second diagram showing the first frame and the second frame to be combined.

FIG. 10 is a diagram showing an example of a panoramic ultrasound image.

FIG. 11 is a diagram showing a reference region.

FIG. 12 is a diagram showing a superimposed portion.

FIG. 13 is a third diagram showing the ultrasound probes located at the first position and the second position.

FIG. 14 is a diagram showing an intermediate frame.

FIG. 15 is a fourth diagram showing the ultrasound probes located at the first position and the second position.

FIG. 16 is a fifth diagram showing the ultrasound probes located at the first position and the second position.

FIG. 17 is a diagram showing a display example of the panoramic ultrasound image and the captured image.

FIG. 18 is a flowchart showing a flow of processing of the ultrasound diagnostic apparatus according to the present embodiment.

### DESCRIPTION OF THE PREFERRED EMBODIMENTS

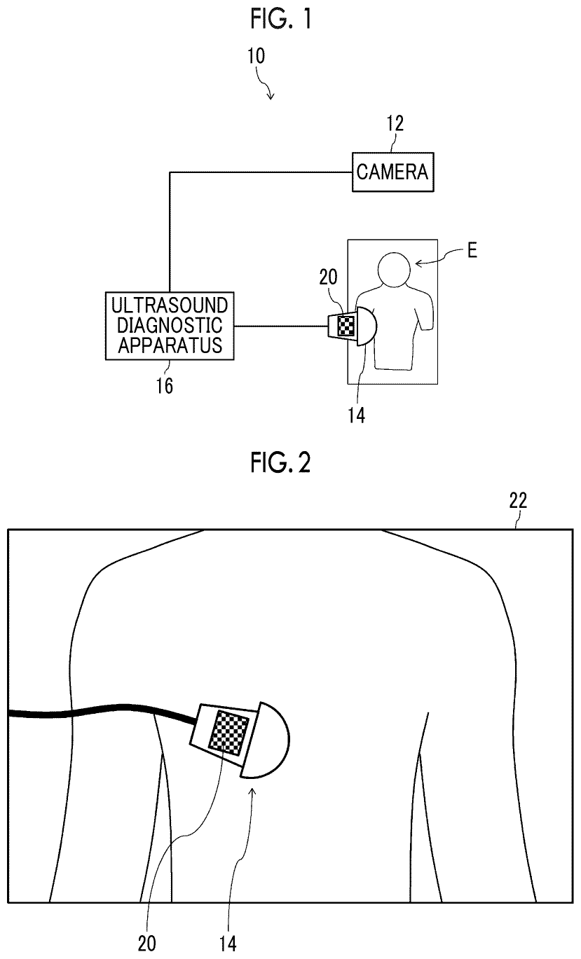

FIG. 1 is a schematic diagram of a configuration of a panoramic ultrasound image forming system 10 according to the present embodiment. The panoramic ultrasound image forming system 10 is configured to include a camera 12 and an ultrasound diagnostic apparatus 16 as a panoramic ultrasound image forming apparatus including an ultrasound probe 14. The camera 12 and the ultrasound diagnostic apparatus 16 are connected to each other in a communicable manner.

A probe detection marker 20 is attached to the ultrasound probe 14. The probe detection marker 20 is a marker for detecting the position and the posture of the ultrasound probe 14. An example of the probe detection marker 20 is an augmented reality (AR) marker.

The camera 12 is configured to include, in addition to a lens and an image sensor, a processor configured of a CPU and the like, a communication interface configured of a network adapter and the like, and the like. The camera 12 images a subject E and the ultrasound probe 14 (specifically, the probe detection marker 20). The camera 12 may be an extracorporeal camera that images a body surface of the subject E, or may be an intracorporeal camera such as an endoscope that is inserted into the subject. A captured image is formed by the image sensor of the camera 12, and the captured image is transmitted to the ultrasound diagnostic apparatus 16 by a communication interface of the camera 12.

FIG. 2 is a diagram showing an example of a captured image 22 of the camera 12. As described above, the captured image 22 includes the image of the probe detection marker 20. The ultrasound diagnostic apparatus 16 can detect at least one of the position or the posture of the ultrasound probe 14 by analyzing the image of the probe detection marker 20 shown in the captured image 22. Details of the detection processing of the position and the posture of the ultrasound probe 14 will be described below.

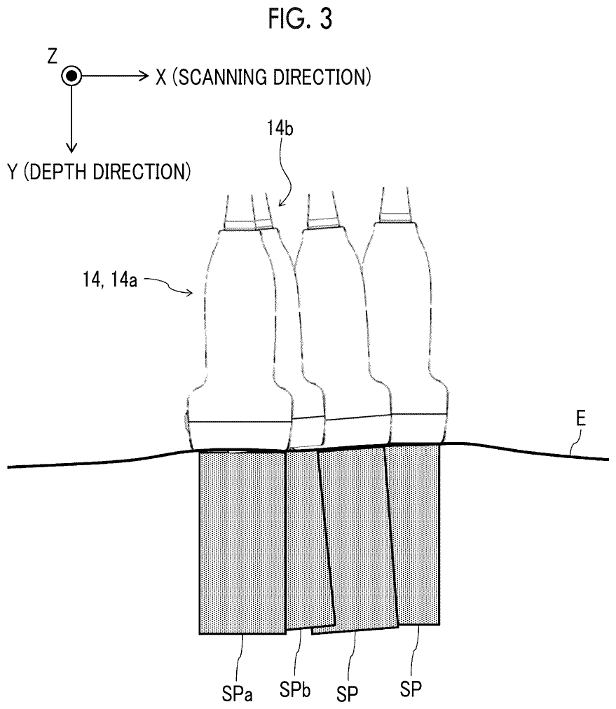

The ultrasound diagnostic apparatus 16 as the panoramic ultrasound image forming apparatus forms a panoramic ultrasound image. FIG. 3 is a diagram showing a plurality of scanning surfaces SP corresponding to a plurality of frames for forming the panoramic ultrasound image. In the drawings of the present specification, including FIG. 3, a horizontal direction in which a scanning direction of the ultrasound beam emitted from the ultrasound probe 14 (in particular, a scanning direction in a case where a first frame among the plurality of frames for forming the panoramic ultrasound image is acquired) is an X-axis, a depth direction is a Y-axis, and a horizontal direction perpendicular to the X-axis and the Y-axis is a Z-axis.

An operator of the ultrasound diagnostic apparatus 16, such as a doctor, forms a plurality of frames corresponding to each scanning surface SP arranged in a scanning direction of the ultrasound beam while moving the ultrasound probe 14 in the scanning direction (X-axis direction) of the ultrasound beam while bringing the ultrasound probe 14 into contact with the subject E. Specifically, the operator positions the ultrasound probe 14 at a first position 14a and scans the scanning surface SPa with the ultrasound beam to form the first frame corresponding to the scanning surface SPa. Next, the ultrasound probe 14 is positioned at a second position 14b spaced from the first position 14a in the scanning direction to scan the scanning surface SPb with the ultrasound beam, thereby forming the second frame corresponding to the scanning surface SPb. Similarly, a plurality of frames corresponding to each scanning surface SP are formed.

The ultrasound diagnostic apparatus 16 forms a panoramic ultrasound image by combining the plurality of frames including the first frame and the second frame. Details of the processing of forming the panoramic ultrasound image will be described below.

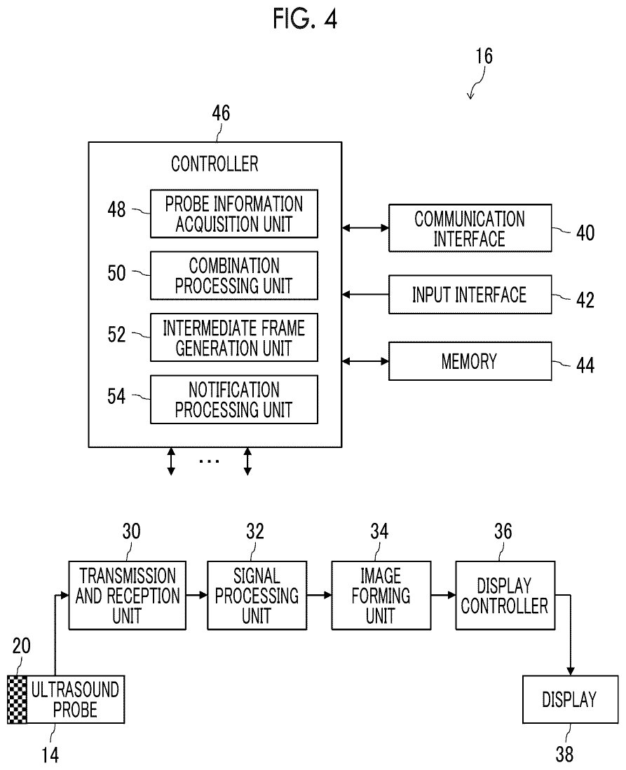

FIG. 4 is a schematic diagram of a configuration of the ultrasound diagnostic apparatus 16. The ultrasound diagnostic apparatus 16 is a medical apparatus installed in medical institutions such as a hospital.

The ultrasound probe 14 is a device that transmits and receives ultrasonic waves to and from the subject E. The ultrasound probe 14 includes a vibration element array consisting of a plurality of vibration elements that transmit and receive ultrasonic waves to and from the subject E. The vibration element array is formed of a plurality of vibration elements arranged in one direction (array direction). A transmission signal is supplied to each vibration element from a transmission and reception unit 30 described below, so that each vibration element generates ultrasonic waves. Specifically, the ultrasound probe 14 scans the ultrasound beam on a plane (scanning surface SP) parallel to the array direction.

As described above, the probe detection marker 20 is attached to the ultrasound probe 14.

The transmission and reception unit 30 transmits the transmission signal to the ultrasound probe 14 (specifically, each vibration element of the vibration element array) under a control of a controller 46 (which will be described below). As a result, the ultrasound beam is scanned from the ultrasound probe 14 on the scanning surface SP. In addition, the transmission and reception unit 30 receives a reception signal from each vibration element that has received a reflected wave from the subject E. The transmission and reception unit 30 includes an adder and a plurality of delayers corresponding to each vibration element, and performs phase alignment and addition processing of aligning and adding phases of the reception signals from each vibration element by the adder and the plurality of delayers. As a result, a reception beam signal is formed in which information indicating a signal intensity of the reflected wave from the subject E is arranged in a depth direction of the subject.

A signal processing unit 32 executes various types of signal processing including filter processing of applying a bandpass filter, detection processing, and the like, on the reception beam signal from the transmission and reception unit 30.

An image forming unit 34 forms an ultrasound tomographic image (B-mode image) representing a cross section (particularly, a scanning surface of the ultrasound beam) of the subject E on the basis of the reception beam signal subjected to the signal processing in the signal processing unit 32. The ultrasound tomographic image formed by the image forming unit 34 is also used as a frame for forming the panoramic ultrasound image.

A display controller 36 performs control to display various images including the ultrasound tomographic image formed by the image forming unit 34 or a panoramic ultrasound image described below on a display 38.

The display 38 as a display unit is, for example, a display device configured using a liquid-crystal display, an organic electro luminescence (EL), or the like.

The transmission and reception unit 30, the signal processing unit 32, the image forming unit 34, and the display controller 36 included in the ultrasound diagnostic apparatus 16 are configured by a processor. The processor is configured to include at least one of a general-purpose processing device (for example, a CPU or the like) or a dedicated processing device (for example, a graphics processing unit (GPU), an application-specific integrated circuit (ASIC), a field-programmable gate array (FPGA), or a programmable logic device). The processor need not be configured using a single processing device but rather may be configured through the collaboration of a plurality of processing devices that are present at physically separated positions. Additionally, each of the above-described units may be implemented through the collaboration of hardware, such as a processor, and software.

A communication interface 40 is configured using, for example, a network adapter or the like. The communication interface 40 exhibits a function of communicating with another device (particularly, the camera 12). In particular, the communication interface 40 receives the captured image from the camera 12.

An input interface 42 is configured using, for example, a button, a trackball, a touch panel, and the like. The input interface 42 is used to input a command of an operator who uses the ultrasound diagnostic apparatus 16, to the ultrasound diagnostic apparatus 16.

A memory 44 is configured to include a hard disk drive (HDD), a solid-state drive (SSD), an embedded MultiMediaCard (eMMC), a read-only memory (ROM), a random-access memory (RAM), or the like. A panoramic ultrasound image forming program for operating each unit of the ultrasound diagnostic apparatus 16 is stored in the memory 44. The panoramic ultrasound image forming program can also be stored in, for example, a computer readable non-transitory storage medium such as a Universal Serial Bus (USB) memory or a CD-ROM. The ultrasound diagnostic apparatus 16 can read and execute the panoramic ultrasound image forming program from such a storage medium. The ultrasound diagnostic apparatus 16 reads the panoramic ultrasound image forming program to exhibit the functions described below, and thus it can be said that the ultrasound diagnostic apparatus 16 is a computer program product.

The controller 46 is configured to include at least one of a general-purpose processor (such as a CPU, for example) or a dedicated processor (such as a GPU, an ASIC, an FPGA, or a programmable logic device, for example). The controller 46 may be configured using a plurality of processing devices that are present at physically separated positions and that collaborate with each other, rather than being configured using a single processing device. The controller 46 controls each unit of the ultrasound diagnostic apparatus 16. In addition, as shown in FIG. 4, the controller 46 exerts functions as a probe information acquisition unit 48, a combination processing unit 50, an intermediate frame generation unit 52, and a notification processing unit 54 in accordance with the panoramic ultrasound image forming program stored in the memory 44.

The probe information acquisition unit 48 acquires positional information indicating the position of the ultrasound probe 14. In particular, the probe information acquisition unit 48 acquires a plurality of pieces of positional information indicating the position of the ultrasound probe 14 in a case where the reception signal corresponding to each frame for forming the panoramic ultrasound image is acquired. In addition, the probe information acquisition unit 48 acquires posture information indicating the posture of the ultrasound probe 14. In particular, the probe information acquisition unit 48 acquires a plurality of pieces of posture information indicating the posture of the ultrasound probe 14 in a case where the reception signal corresponding to each frame for forming the panoramic ultrasound image is acquired.

In the present embodiment, the probe information acquisition unit 48 acquires the positional information and the posture information by analyzing the captured image 22 acquired by the camera 12 to detect the position and the posture of the ultrasound probe 14. As described above, the captured image 22 includes the image of the probe detection marker 20 indicating the position and the posture of the ultrasound probe 14 (see FIG. 2). The probe information acquisition unit 48 acquires the positional information and the posture information by analyzing the image of the probe detection marker 20 in the captured image 22. The positional information may be, for example, three-dimensional coordinates in a camera coordinate system. The posture information may be a rotation angle with respect to a predetermined axis (for example, the X-axis, the Y-axis, or the Z-axis) in the camera coordinate system. Since a known method can be used as a method of detecting the position and the posture of the ultrasound probe 14 in the camera coordinate system from the image of the probe detection marker 20 included in the captured image 22, the detailed description thereof will be omitted here.

The probe information acquisition unit 48 executes calibration prior to detecting the position and the posture of the ultrasound probe 14 based on the captured image 22. Specifically, the operator sets the position and the posture of the ultrasound probe 14 to predetermined position and posture, and inputs the calibration instruction to the ultrasound diagnostic apparatus 16 in that state. The probe information acquisition unit 48 detects the position and the posture of the ultrasound probe 14 in a case where the calibration instruction is input, on the basis of the probe detection marker 20 included in the captured image 22, and stores the position and the posture in the memory 44 as a reference position and a reference posture. Thereafter, the probe information acquisition unit 48 detects the position of the ultrasound probe 14 as a relative position with respect to a known reference position, and detects the posture of the ultrasound probe 14 as a relative posture with respect to a known reference posture.

In the present embodiment, it is assumed that the operator inputs the calibration instruction at the position and the posture of the ultrasound probe 14 in a case where the first frame among the plurality of frames for forming the panoramic ultrasound image is acquired, and the position and the posture of the ultrasound probe 14 at that time are the reference position and the reference posture. Of course, the position and the posture of the ultrasound probe 14 in a case of performing the calibration are not limited thereto.

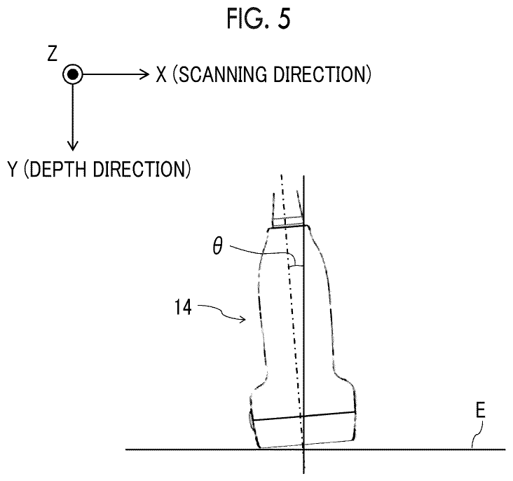

FIG. 5 is a diagram showing an angle θ as the posture information. For example, in a case where the calibration is performed in a posture in which the ultrasound probe 14 is brought into contact with the surface of the subject E perpendicularly (in other words, in a state in which the scanning direction of the ultrasound beam is parallel to the surface of the subject E), the inclination of the ultrasound probe 14 in an XY plane can be represented by the angle θ. As described above, the posture information can be represented by a rotation angle in each of the XY plane (around the Z-axis), a YZ plane (around the X-axis), and an XZ plane (around the Y-axis) with respect to the posture of the ultrasound probe 14 in a case of the calibration.

The probe information acquisition unit 48 may detect at least one of the positional information or the posture information of the ultrasound probe 14 via a method other than analyzing the captured image 22. For example, a position and posture sensor such as a magnetic sensor or an acceleration sensor may be provided in the ultrasound probe 14, and at least one of the positional information or the posture information may be detected based on a detection value of the position and posture sensor.

The scanning surface SP (see FIG. 3) is determined by the position and the posture of the ultrasound probe 14. Therefore, it can be said that the positional information and the posture information acquired by the probe information acquisition unit 48 indicate the position and the posture of the scanning surface SP corresponding to each frame for forming the panoramic ultrasound image.

The probe information acquisition unit 48 stores, in the memory 44, the positional information indicating the position of the ultrasound probe 14 in a case where the transmission and reception unit 30 acquires the reception signal corresponding to each frame, and the posture information indicating the posture of the ultrasound probe 14 in a case where the transmission and reception unit 30 acquires the reception signal corresponding to each frame, in association with the reception signal or the frame formed from the reception signal.

The combination processing unit 50 combines a plurality of frames to form a panoramic ultrasound image. The combination processing unit 50 can form a panoramic ultrasound image by combining three or more frames, but in the following, processing of combining two frames (a first frame and a second frame) will be described. After a third frame (that is, the combination of the second frame and the third frame, the combination of the third frame and a fourth frame, . . . ), the combination processing of the first frame and the second frame can be performed in the same manner.

In the present specification, a position of the ultrasound probe 14 in a case where the reception signal corresponding to the first frame is acquired is referred to as a first position, a posture of the ultrasound probe 14 in a case where the reception signal corresponding to the first frame is acquired (in other words, in a case where the ultrasound probe 14 is located at the first position) is referred to as a first posture, a position of the ultrasound probe 14 in a case where the reception signal corresponding to the second frame is acquired and which is different from the first position is referred to as a second position, and a posture of the ultrasound probe 14 in a case where the reception signal corresponding to the second frame is acquired (in other words, in a case where the ultrasound probe 14 is located at the second position) is referred to as a second posture.

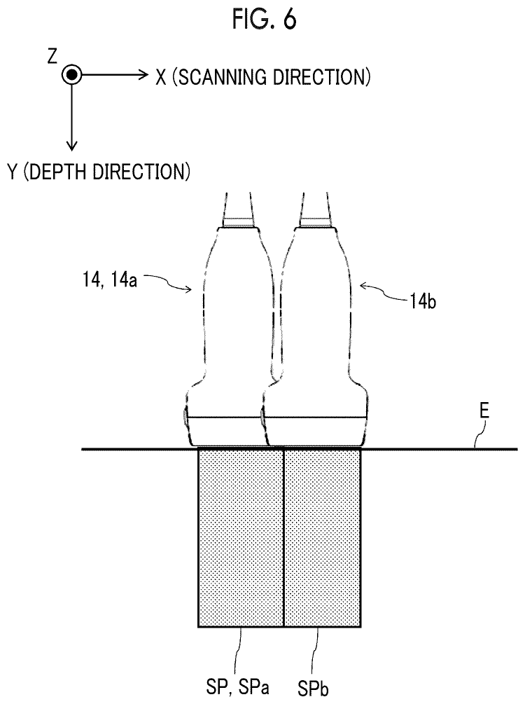

FIG. 6 is a first diagram showing the ultrasound probe 14 located at the first position 14a and the second position 14b. In the example of FIG. 6, the surface of the subject E on which the ultrasound probe 14 is moved is flat, and there is almost no change in the posture of the ultrasound probe 14 in a case where the ultrasound probe 14 is moved to acquire a plurality of frames. In this case, the combination processing unit 50 can combine the plurality of frames based on the positional information of the ultrasound probe 14.

Specifically, the combination processing unit 50 combines the first frame and the second frame by shifting (translating) the first frame and the second frame based on a difference between the first position 14a indicated by the positional information acquired by the probe information acquisition unit 48 and the second position 14b indicated by the positional information acquired by the probe information acquisition unit 48 (the second position 14b is in the scanning direction of the ultrasound beam from the first position 14a). The difference (a distance) between the first position 14a and the second position 14b indicated by the positional information is a distance in the camera coordinate system or the real space. On the other hand, as described above, the position of the scanning surface SP is determined by the position of the ultrasound probe 14, and the size thereof (particularly, the width in the scanning direction) is also known. In addition, the number of pixels of the frame corresponding to the scanning surface SP (particularly, the number of pixels in the scanning direction) is predetermined. Therefore, the combination processing unit 50 can calculate the distance in the camera coordinate system or the real space per unit pixel of the frame in the scanning direction.

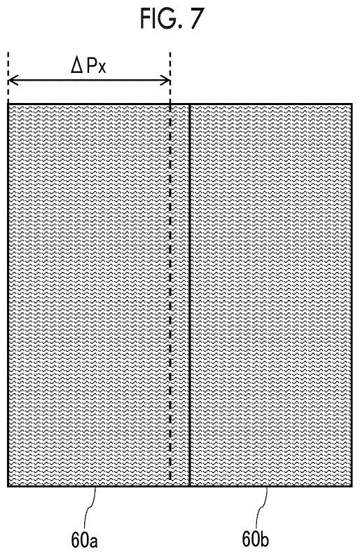

FIG. 7 is a diagram showing positions of a first frame 60a and a second frame 60b to be combined. The combination processing unit 50 calculates a positional relationship in the scanning direction (generally, a lateral direction of the image) between the first frame 60a and the second frame 60b corresponding to the positional relationship in the scanning direction between the scanning surface SPa and the scanning surface SPb, based on the distance in the camera coordinate system or the real space per unit pixel of the frame in the scanning direction and the difference between the first position 14a and the second position 14b (in other words, the difference in position between the scanning surface SPa and the scanning surface SPb). Specifically, the combination processing unit 50 calculates the difference (ΔPx) in the position in the scanning direction between the first frame 60a and the second frame 60b in terms of the number of pixels. Then, the combination processing unit 50 disposes the second frame 60b by shifting the second frame 60b in the scanning direction by the calculated difference ΔPx with respect to the first frame 60a, and then combines the first frame 60a and the second frame 60b.

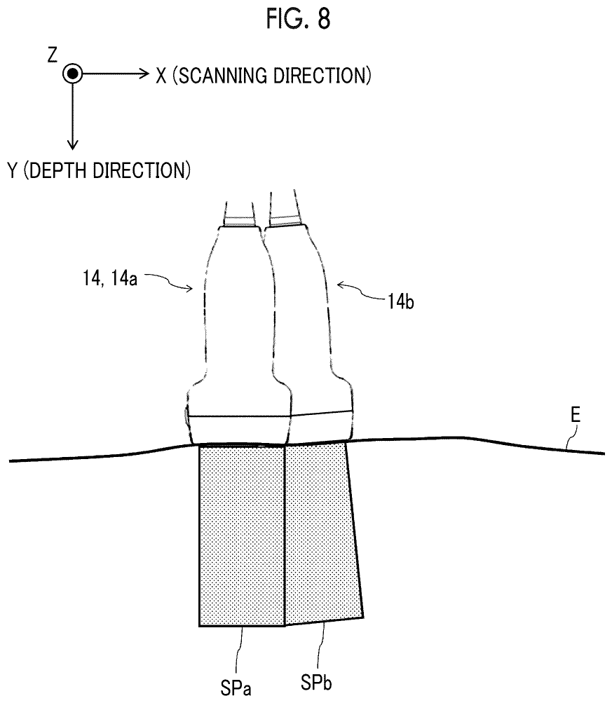

FIG. 8 is a second diagram showing the ultrasound probes 14 located at the first position 14a and the second position 14b. In the example of FIG. 8, the surface of the subject E on which the ultrasound probe 14 is moved has irregularities, and the posture of the ultrasound probe 14 changes in a case where the ultrasound probe 14 is moved to acquire a plurality of frames. In this case, the combination processing unit 50 combines the plurality of frames based on the positional information and the posture information of the ultrasound probe 14.

Specifically, the combination processing unit 50 rotates the second frame 60b relative to the first frame 60a in accordance with a rotation amount corresponding to a difference between the first posture indicated by the posture information acquired by the probe information acquisition unit 48 and the second posture indicated by the posture information acquired by the probe information acquisition unit 48, and combines the first frame 60a and the second frame 60b by shifting the first frame 60a and the second frame 60b based on the difference between the first position 14a and the second position 14b. As described above, since the posture of the scanning surface SP is determined by the posture of the ultrasound probe 14, the combination processing unit 50 can obtain the difference in the posture between the scanning surface SPa and the scanning surface SPb based on the difference between the first posture and the second posture (in other words, the difference in the position between the scanning surface SPa and the scanning surface SPb).

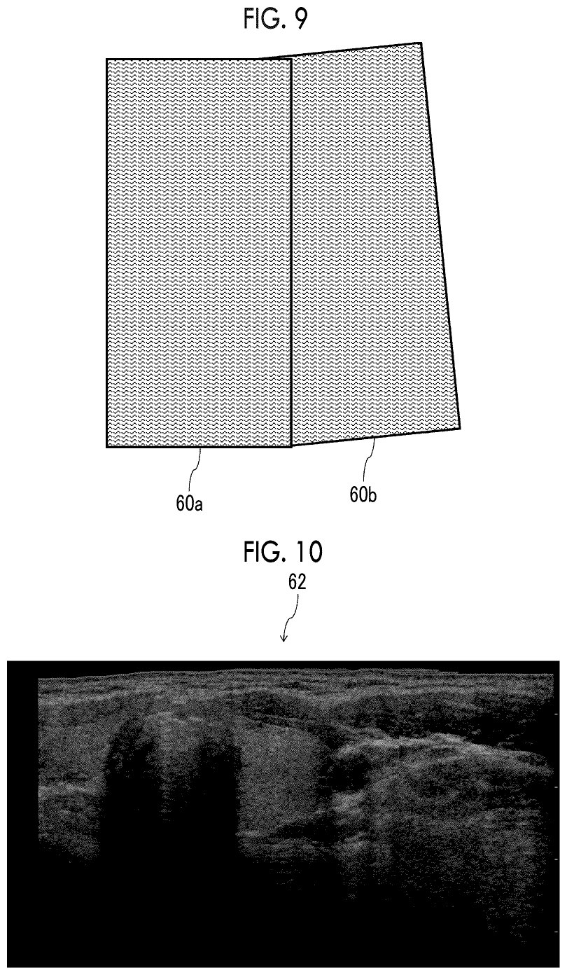

FIG. 9 is a diagram showing positions and postures of the first frame 60a and the second frame 60b to be combined. As shown in FIGS. 8 and 9, the combination processing unit 50 relatively rotates the second frame 60b with respect to the first frame 60a such that the posture of the scanning surface SPb is the same as the posture of the scanning surface SPa. In addition, the combination processing unit 50 disposes the second frame 60b by shifting the second frame 60b in the scanning direction by the calculated difference ΔPx with respect to the first frame 60a, and then combines the first frame 60a and the second frame 60b.

FIG. 10 shows an example of a panoramic ultrasound image 62 formed by the combination processing unit 50. As described above, in the present embodiment, the combination processing unit 50 determines the position of the second frame 60b with respect to the first frame 60a or the position and the posture based on the first position 14a and the second position 14b of the ultrasound probe 14 acquired by the probe information acquisition unit 48, and combines the first frame 60a and the second frame 60b. Therefore, in the present embodiment, since the first frame 60a and the second frame 60b are not registered on the basis of the similarity between the frames, the accuracy of the registration between the first frame 60a and the second frame 60b can be improved, and thus the image quality of the panoramic ultrasound image 62 can be improved. In addition, in the present embodiment, since the first frame 60a and the second frame 60b are registered on the basis of the first position 14a and the second position 14b without the need to calculate the similarity between the frames for the registration of the first frame 60a and the second frame 60b, the amount of calculation for the registration (that is, the formation of the panoramic ultrasound image 62) of the first frame 60a and the second frame 60b can be reduced.

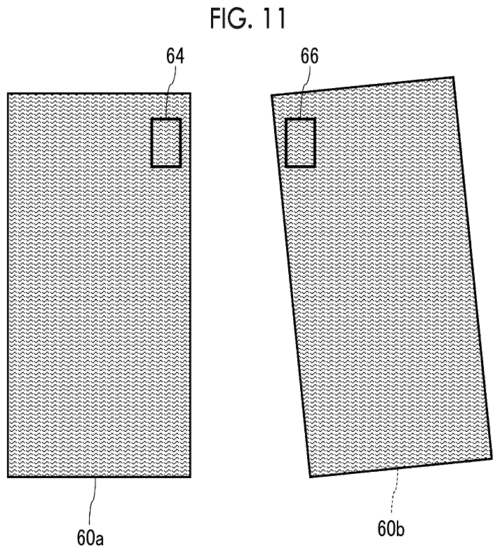

FIG. 11 is a diagram showing a reference region 64 set in the first frame 60a. In the present embodiment, in addition to the first position 14a and the second position 14b of the ultrasound probe 14, the position of the second frame 60b with respect to the first frame 60a or the position and the posture may be determined on the basis of the similarity between the frames between the first frame 60a and the second frame 60b. Specifically, the combination processing unit 50 sets a part of the first frame 60a as the reference region 64. Next, the combination processing unit 50 performs image matching processing or the like between the reference region 64 and the second frame 60b to specify a corresponding region 66, which is a region in the second frame 60b corresponding to the reference region 64 (having a similarity equal to or higher than a predetermined similarity threshold value). Then, the combination processing unit 50 combines the first frame 60a and the second frame 60b by shifting the first frame 60a and the second frame 60b based on the difference between the reference region 64 and the corresponding region 66. Specifically, the combination processing unit 50 determines the position of the second frame 60b with respect to the first frame 60a or the position and the posture such that the reference region 64 and the corresponding region 66 overlap each other.

In the present embodiment, since the first position 14a and the second position 14b of the ultrasound probe 14 are also used, for example, after the position of the second frame 60b with respect to the first frame 60a or the position and the posture is determined based on the first position 14a and the second position 14b, in a case where the reference region 64 and the corresponding region 66 are deviated from each other, processing of finely adjusting the position of the second frame 60b with respect to the first frame 60a or the position and the posture such that the reference region 64 and the corresponding region 66 overlap each other can be performed.

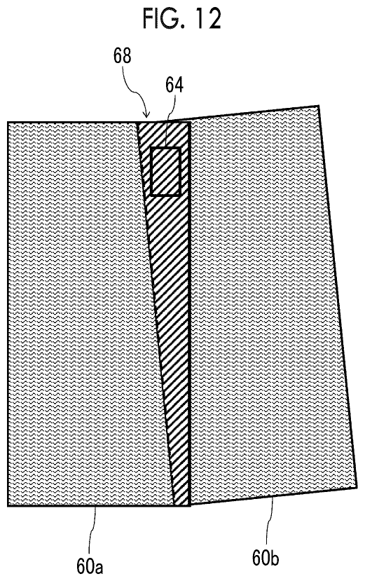

FIG. 12 is a diagram showing a superimposed portion 68. Here, the reference region 64 needs to be set in the superimposed portion 68 (hatched portion in FIG. 12) that is a portion of the first frame 60a corresponding to a portion where the scanning surface SPa corresponding to the first frame 60a and the scanning surface SPb corresponding to the second frame 60b overlap each other. This is because, in a case where the reference region 64 is set in a portion other than the superimposed portion 68, there should be no region in the second frame 60b in which the similarity with the reference region 64 is equal to or higher than the similarity threshold value, and the corresponding region 66 is not detected from the second frame 60b. In the present embodiment, the combination processing unit 50 can specify the portion where the scanning surface SPa and the scanning surface SPb overlap each other, based on the first position 14a and the second position 14b acquired by the probe information acquisition unit 48. Therefore, the combination processing unit 50 sets the reference region 64 in the superimposed portion 68 based on the first position 14a and the second position 14b.

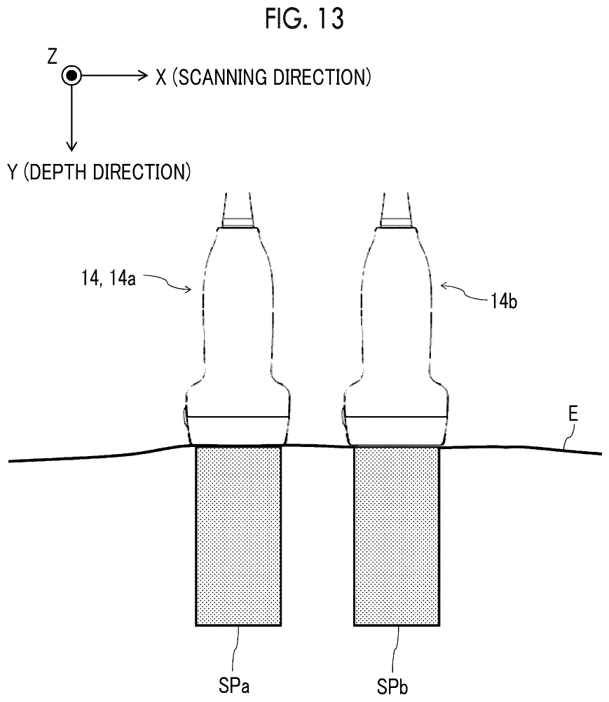

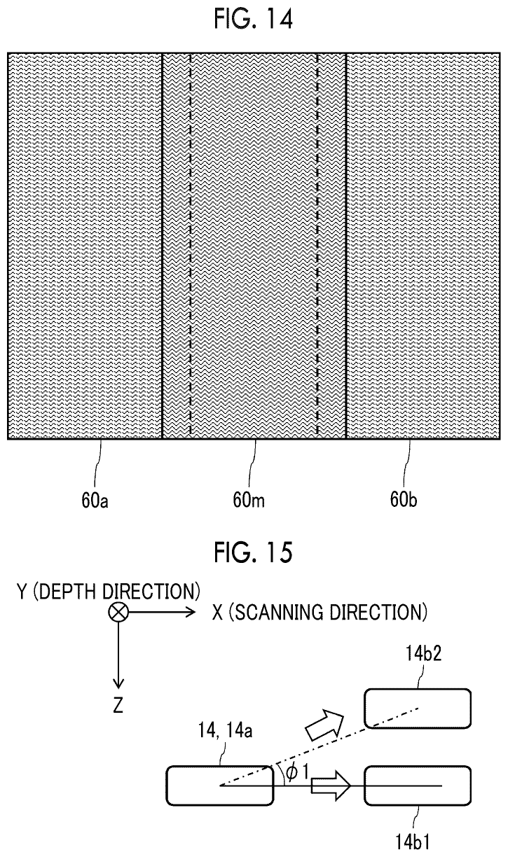

The processing of the intermediate frame generation unit 52 will be described with reference to FIGS. 13 and 14. FIG. 13 is a third diagram showing the ultrasound probe 14 located at the first position 14a and the second position 14b. FIG. 14 is a diagram showing an intermediate frame 60m. In a case where the operator quickly moves the ultrasound probe 14 in the scanning direction in a case of acquiring a plurality of frames for forming the panoramic ultrasound image 62, the first position 14a and the second position 14b may be separated from each other as shown in FIG. 13. In such a case, there may be no superimposed portion between the scanning surface SPa and the scanning surface SPb, and there may be a gap between the scanning surface SPa and the scanning surface SPb. In a case where the first frame 60a corresponding to the scanning surface SPa and the second frame 60b corresponding to the scanning surface SPb are combined as they are, the first frame 60a and the second frame 60b are discontinuous in the panoramic ultrasound image 62.

Therefore, in a case where the second position 14b is separated from the first position 14a by a distance equal to or greater than the first threshold distance, the intermediate frame generation unit 52 generates the intermediate frame 60m that interpolates between the first frame 60a and the second frame 60b in the panoramic ultrasound image 62. The first threshold distance may be predetermined. For example, the first threshold distance is set to a distance between the first position 14a and the second position 14b such that the scanning surface SPa and the scanning surface SPb do not overlap each other.

Specifically, the intermediate frame generation unit 52 generates the intermediate frame 60m based on the first frame 60a and the second frame 60b. A known technology can be used as a method of generating the intermediate frame 60m based on the first frame 60a and the second frame 60b. For example, the intermediate frame 60m can be generated by applying a spatio-temporal filter to the first frame 60a and the second frame 60b. As the filter type, a smoothing filter, an anisotropic filter, an application filter, or a combination thereof can be used. In addition, the intermediate frame 60m may be non-linearly corrected by detecting the local change of the second frame 60b with respect to the first frame 60a.

In a case where the intermediate frame 60m is generated by the intermediate frame generation unit 52, the combination processing unit 50 combines the first frame 60a, the second frame 60b, and the intermediate frame 60m disposed between the first frame 60a and the second frame 60b to form the panoramic ultrasound image 62. In this case, the intermediate frame 60m may be disposed in the middle between the first frame 60a and the second frame 60b.

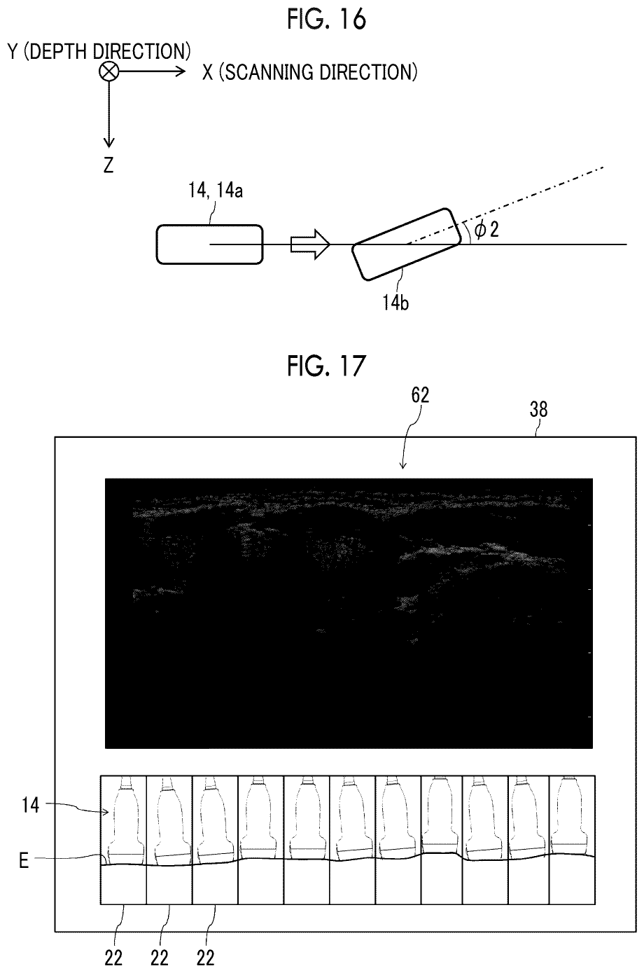

The processing of the notification processing unit 54 will be described with reference to FIGS. 15 and 16. FIG. 15 is a fourth diagram showing the ultrasound probe 14 located at the first position 14a and the second position 14b. FIG. 15 is a plan view of the ultrasound probe 14. In order to appropriately form the panoramic ultrasound image 62, it is necessary for the operator to move the ultrasound probe 14 in the scanning direction in a case of acquiring a plurality of frames. That is, in FIG. 15, the ultrasound probe 14 may be moved from the first position 14a to the second position 14b1 in the scanning direction, but in a case where the second position 14b2 is a position to which the first position 14a has transitioned in the Z-axis direction, it is not possible to form an appropriate panoramic ultrasound image 62.

Therefore, in a case where an angle φ1 formed between the scanning direction (X-axis direction in the example of FIG. 15) of the ultrasound beam from the ultrasound probe 14 in a case where the ultrasound probe 14 is located at the first position 14a and the direction from the first position 14a toward the second position 14b is equal to or greater than a predetermined first threshold angle, the notification processing unit 54 may notify the operator. The notification method can be performed, for example, by displaying a warning on the display 38, outputting a warning audio, causing a light emitting unit to emit light, or a combination thereof.

FIG. 16 is a fifth diagram showing the ultrasound probe 14 located at the first position 14a and the second position 14b. FIG. 16 is also a plan view of the ultrasound probe 14. In order to appropriately form the panoramic ultrasound image 62, it is also necessary to keep the scanning direction of the ultrasound beam in the ultrasound probe 14 constant in a case where a plurality of frames are acquired. In a case where the scanning direction of the ultrasound beam fluctuates while a plurality of frames are acquired, it is not possible to form an appropriate panoramic ultrasound image 62.

Therefore, in a case where an angle φ2 formed between the scanning direction (X-axis direction in the example of FIG. 16) of the ultrasound beam in the first posture, which is the posture of the ultrasound probe 14 in a case where the ultrasound probe 14 is located at the first position 14a, and the scanning direction of the ultrasound beam in the second posture, which is the posture of the ultrasound probe 14 in a case of where the ultrasound probe 14 is located at the second position 14b, is equal to or greater than a predetermined second threshold angle, the notification processing unit 54 may notify the operator.

In addition, as shown in FIG. 13, in a case where the first position 14a and the second position 14b are separated from each other by a distance equal to or greater than the first threshold distance, the intermediate frame generation unit 52 generates the intermediate frame 60m, but from the viewpoint of forming an appropriate panoramic ultrasound image 62, it is undesirable that the first position 14a and the second position 14b are separated from each other in the first place.

Therefore, the notification processing unit 54 may notify the operator in a case where the second position 14b is separated from the first position 14a by a distance equal to or greater than the second threshold distance. Here, the second threshold distance may be the same as or different from the first threshold distance.

FIG. 17 is a diagram showing a display example of the panoramic ultrasound image 62 and the captured image 22. The display controller 36 displays the panoramic ultrasound image 62 formed by the combination processing unit 50 on the display 38. In addition, the display controller 36 may display the panoramic ultrasound image 62 together with the plurality of captured images 22 obtained by imaging the ultrasound probe 14 and the subject E, the captured images 22 being arranged in an imaging order, in a case where the reception signals corresponding to each of the plurality of frames for forming the panoramic ultrasound image 62 are acquired. In particular, as shown in FIG. 17, the display controller 36 may display the plurality of captured images 22 side by side in the left and right directions in the imaging order such that each frame constituting the panoramic ultrasound image 62 and the captured image 22 captured in a case where the reception signal corresponding to the frame is acquired correspond to each other above or below the panoramic ultrasound image 62. As a result, the operator can easily ascertain which position of the subject E each frame constituting the panoramic ultrasound image 62 corresponds to, or the position and the posture of the ultrasound probe 14 in a case where each frame is acquired.

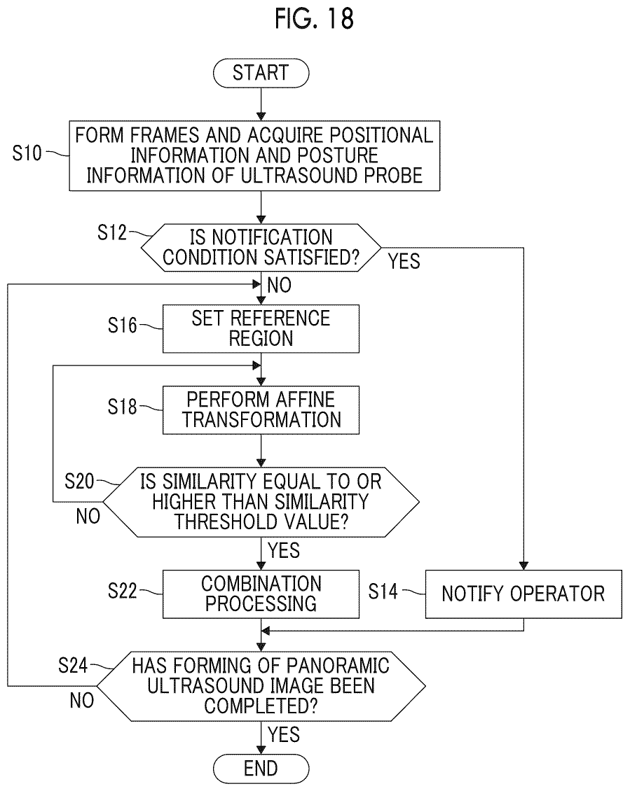

The outline of the configuration of the panoramic ultrasound image forming system 10 according to the present embodiment is as described above. Hereinafter, a flow of the processing of the ultrasound diagnostic apparatus 16 will be described with reference to a flowchart shown in FIG. 18.

In step S10, the operator moves the ultrasound probe 14 in the scanning direction of the ultrasound beam. The image forming unit 34 forms a plurality of frames corresponding to each scanning surface SP arranged in the scanning direction of the ultrasound beam based on the reception signal acquired by the ultrasound probe 14 transmitting and receiving the ultrasonic waves to and from the subject E. During this time, the camera 12 images the ultrasound probe 14 (specifically, the probe detection marker 20) and transmits the captured image 22 to the ultrasound diagnostic apparatus 16. The probe information acquisition unit 48 acquires a plurality of pieces of positional information indicating the position of the ultrasound probe 14 in a case where the reception signal corresponding to each frame is acquired, and a plurality of pieces of posture information indicating the posture of the ultrasound probe 14 in a case where the reception signal corresponding to each frame is acquired, by analyzing the captured image 22.

In step S12, the notification processing unit 54 determines whether or not the position of the ultrasound probe 14 in a case where the reception signals corresponding to two adjacent frames in the acquisition order are acquired or the posture of the ultrasound probe 14 satisfies the notification condition, on the basis of the positional information and the posture information acquired in step S10. For example, the notification processing unit 54 determines whether or not the angle φ1 (refer to FIG. 15) formed between the scanning direction of the ultrasound beam from the ultrasound probe 14 in a case where the ultrasound probe 14 is located at the first position 14a and the direction from the first position 14a toward the second position 14b is equal to or greater than the predetermined first threshold angle. Alternatively, the notification processing unit 54 determines whether or not the angle φ2 (see FIG. 16) formed between the scanning direction of the ultrasound beam in the first posture and the scanning direction of the ultrasound beam in the second posture is equal to or greater than the predetermined second threshold angle. Alternatively, it is determined whether or not the second position 14b is separated from the first position 14a by a distance equal to or greater than the second threshold distance. In a case where it is determined that the notification condition is satisfied, the process proceeds to step S14.

In step S14, the notification processing unit 54 notifies the operator. In a case where it is determined in step S12 that the notification condition is not satisfied, the process proceeds to step S16.

In step S16, the combination processing unit 50 sets the reference region 64 in the first frame 60a based on the positional information and the posture information acquired in step S10. The combination processing unit 50 sets the reference region 64 in the superimposed portion 68 (see FIG. 12) based on the first position 14a and the second position 14b.

In step S18, the combination processing unit 50 performs an affine transformation of the second frame 60b based on the positional information and the posture information acquired in step S10. Specifically, the combination processing unit 50 calculates a difference (ΔPx) in the position in the scanning direction between the first frame 60a and the second frame 60b in terms of the number of pixels, which corresponds to the positional relationship in the scanning direction between the scanning surface SPa and the scanning surface SPb, based on the difference between the first position 14a and the second position 14b, and moves the second frame 60b in the scanning direction by the calculated difference ΔPx while remaining parallel with respect to the first frame 60a. In addition, the combination processing unit 50 relatively rotates the second frame 60b with respect to the first frame 60a such that the posture of the scanning surface SPb is the same as the posture of the scanning surface SPa based on the difference between the first posture and the second posture.

In step S20, the combination processing unit 50 specifies the corresponding region 66 in the second frame 60b corresponding to the reference region 64 set in step S16, and determines whether or not the similarity between the reference region 64 and the corresponding region 66 is equal to or higher than a predetermined similarity threshold value. In a case where the similarity between the reference region 64 and the corresponding region 66 is less than a predetermined similarity threshold value, the process returns to step S18, and the combination processing unit 50 adjusts the position or the posture of the second frame 60b such that the similarity between the reference region 64 and the corresponding region 66 is equal to or higher than the predetermined similarity threshold value. In a case where the similarity between the reference region 64 and the corresponding region 66 is equal to or higher than a predetermined similarity threshold value, the process proceeds to step S22.

In step S22, the combination processing unit 50 combines the first frame 60a and the second frame 60b subjected to the affine transformation in step S18.

In step S24, the combination processing unit 50 determines whether or not the combination processing is completed for all the frames for forming the panoramic ultrasound image 62. In a case where there is still a remaining frame, the process returns to step S16, and the processing of steps S16 to S22 is repeated for the next frame (for example, the second frame 60b and the third frame). In a case where it is determined in step S24 that the combination processing is completed for all the frames for forming the panoramic ultrasound image 62, the process ends.

Although the panoramic ultrasound image forming apparatus according to the present disclosure has been described above, the panoramic ultrasound image forming apparatus according to the present disclosure is not limited to the above-described embodiment, and various modifications can be made without departing from the scope of the present disclosure.

In addition, for example, in each of the above-described embodiments, the panoramic ultrasound image forming apparatus is the ultrasound diagnostic apparatus 16, and each of the functions of the image forming unit 34, the display controller 36, the probe information acquisition unit 48, the combination processing unit 50, the intermediate frame generation unit 52, and the notification processing unit 54 is provided in the ultrasound diagnostic apparatus 16. However, each of these functions may not necessarily be performed by the ultrasound diagnostic apparatus 16. For example, these functions may be performed by a server computer or the like that is communicably connected to the ultrasound diagnostic apparatus 16. In addition, all of the above-described functions may not be performed by one device, and the above-described functions may be performed by cooperation of a plurality of devices.

What is claimed is:

1. A panoramic ultrasound image forming apparatus that forms a panoramic ultrasound image by combining a plurality of frames including a first frame obtained by scanning with an ultrasound beam from an ultrasound probe located at a first position toward a subject and a second frame obtained by scanning with the ultrasound beam from the ultrasound probe located at a second position different from the first position toward the subject, comprising:
a probe information acquisition unit that acquires positional information indicating a position of the ultrasound probe in a case where a reception signal corresponding to each frame for forming the panoramic ultrasound image is acquired; and
a combination processing unit that forms the panoramic ultrasound image by combining the plurality of frames, the combination processing unit being configured to combine the first frame and the second frame by shifting the first frame and the second frame based on a difference between the first position and the second position located in a scanning direction of the ultrasound beam from the first position.

2. The panoramic ultrasound image forming apparatus according to claim 1,
wherein the probe information acquisition unit further acquires posture information indicating a posture of the ultrasound probe in a case where the reception signal corresponding to each frame for forming the panoramic ultrasound image is acquired, and
the combination processing unit rotates the second frame with respect to the first frame in accordance with a rotation amount corresponding to a difference between a first posture, which is a posture of the ultrasound probe in a case where the ultrasound probe is located at the first position, and a second posture, which is a posture of the ultrasound probe in a case where the ultrasound probe is located at the second position, and combines the first frame and the second frame.

3. The panoramic ultrasound image forming apparatus according to claim 1,
wherein the combination processing unit
sets, based on the first position and the second position, a reference region in a superimposed portion that is a portion of the first frame corresponding to a portion where a scanning surface corresponding to the first frame and a scanning surface corresponding to the second frame overlap each other, and
specifies a corresponding region that is a region in the second frame corresponding to the reference region of the first frame and combines the first frame and the second frame by shifting the first frame and the second frame based on a difference between a position of the reference region and a position of the corresponding region.

4. The panoramic ultrasound image forming apparatus according to claim 1, further comprising:
an intermediate frame generation unit that generates an intermediate frame for interpolating between the first frame and the second frame in the panoramic ultrasound image based on the first frame and the second frame in a case where the second position is separated from the first position by a distance equal to or greater than a first threshold distance,
wherein the combination processing unit forms the panoramic ultrasound image by combining the first frame, the second frame, and the intermediate frame disposed between the first frame and the second frame.

5. The panoramic ultrasound image forming apparatus according to claim 1, further comprising:
a notification processing unit that notifies an operator in a case where an angle formed between a scanning direction of the ultrasound beam from the ultrasound probe in a case where the ultrasound probe is located at the first position and a direction from the first position toward the second position is equal to or greater than a first threshold angle.

6. The panoramic ultrasound image forming apparatus according to claim 2, further comprising:
a notification processing unit that notifies an operator in a case where an angle formed between a scanning direction of the ultrasound beam in the first posture and a scanning direction of the ultrasound beam in the second posture is equal to or greater than a second threshold angle.

7. The panoramic ultrasound image forming apparatus according to claim 1, further comprising:
a notification processing unit that notifies an operator in a case where the second position is separated from the first position by a distance equal to or greater than a second threshold distance.

8. The panoramic ultrasound image forming apparatus according to claim 1, further comprising:
a camera that images the subject and the ultrasound probe; and
a display controller that displays, on a display unit, the panoramic ultrasound image together with a plurality of captured images obtained by imaging the ultrasound probe and the subject, the captured images being arranged in an imaging order, in a case where reception signals corresponding to each of the plurality of frames are acquired.

9. A non-transitory computer-readable storage medium storing a panoramic ultrasound image forming program causing a computer that forms a panoramic ultrasound image by combining a plurality of frames including a first frame obtained by scanning with an ultrasound beam from an ultrasound probe located at a first position toward a subject and a second frame obtained by scanning with the ultrasound beam from the ultrasound probe located at a second position different from the first position toward the subject, to function as:
a probe information acquisition unit that acquires positional information indicating a position of the ultrasound probe in a case where a reception signal corresponding to each frame for forming the panoramic ultrasound image is acquired; and
a combination processing unit that forms the panoramic ultrasound image by combining the plurality of frames, the combination processing unit being configured to combine the first frame and the second frame by shifting the first frame and the second frame based on a difference between the first position and the second position located in a scanning direction of the ultrasound beam from the first position.
